(19) United States
(12) Patent Application Publication
(10) Pub. No.: US 2009/0149718 A1
(43) Pub. Date: Jun. 11, 2009

(54) SYSTEM FOR MEASURING BIO-SIGNALS AND METHOD OF PROVIDING HEALTH CARE SERVICE USING THE SAME

(75) Inventors: Young Sung KIM, Daejeon (KR); Kyu Chang KANG, Daejeon (KR); Jeun Woo LEE, Daejeon (KR); Yun Kyung PARK, Daejeon (KR); Ki Ryong HA, Daejeon (KR); Hyung Jik LEE, Daejeon (KR)

Correspondence Address: STAAS & HALSEY LLP, SUITE 700, 1201 NEW YORK AVENUE, N.W., WASHINGTON, DC, 20005, US

(73) Assignee: ELECTRONICS AND TELECOMMUNICATIONS RESEARCH INSTITUTE, Daejeon (KR)

(21) Appl. No.: 12/170,081

(22) Filed: Jul. 9, 2008

(30) Foreign Application Priority Data
Dec. 10, 2007 (KR) 10-2007-0127611

(51) Int. Cl. A61B 5/00

(52) U.S. Cl. 600/300

(57) ABSTRACT

Disclosed herein is a bio-signal measurement system for measuring bio-signals and providing relevant health care service. The bio-signal measurement system includes a bio-signal measurement device and a portable terminal device. The bio-signal measurement device is formed in a necklace shape, and is configured to measure various bio-signals, perform signal processing on the measured bio-signals, and send the processed bio-signals through a short-range wireless communication module. The portable terminal device is configured to perform signal processing on the sent bio-signals, store the processed bio-signals, calculate relevant bio-indexes, provide a monitoring service related to the bio-signals, compare the calculated bio-indexes with previously stored bio-indexes, and selectively provide a relevant health care information service.

### CROSS-REFERENCE(S) TO RELATED APPLICATIONS

The present invention claims priority of Korean Patent Application No. 10-2007-0127611, filed on Dec. 10, 2007, which is incorporated herein by reference.

### FIELD OF THE INVENTION

The present invention relates to a system for measuring bio-signals, and, more particularly, to a system for measuring bio-signals and a method of providing health care service using the system, which are suitable for the provision of health care service through the measurement of bio-signals.

This work was supported by the IT R&D program of MIC/IITA. [2005-S-069-03, Wearable System Using Physiological Signal Processing]

### BACKGROUND OF THE INVENTION

As is well known, “ubiquitous network” refers to an information and communication environment that enables users to freely access networks without being concerned about networks or computers and regardless of location. Technologies related to such a ubiquitous network can be applied to almost all fields of human life.

In particular, recently, due to the well-being trend, ubiquitous health care (U-health care) gets attracting attention as a noteworthy technical field. Ubiquitous health care refers to ubiquitous technology for installing chips or sensors related to medical service throughout humans' living areas and enabling all humans to be effortlessly provided with medical service anytime and anywhere. According to such ubiquitous health care, medical activities, such as medical checkups, disease management, emergency management and interviews with a doctor, which have heretofore been performed only in hospitals, can be effortlessly performed in daily life without visiting hospitals.

For example, a diabetic wears a blood sugar management belt in which a blood sugar management program has been installed, the diabetic's blood sugar is frequently measured by using a blood sugar sensor attached to the belt, an amount of insulin in conformity with the measured blood sugar level can be calculated, information about blood sugar can be provided to the physician in charge over a wireless communication network if the diabetic's blood sugar abruptly decreases or increases, and the physician in charge, who has received the information about blood sugar, can make an optimal prescription or take measures to respond to an emergency situation.

As an example of such ubiquitous health care, portable devices capable of measuring various bio-signals, such as an electrocardiogram, body fat and a sphygmogram, has been developed and used recently.

However, the prior art devices can measure bio-signals, such as a sphygmogram and an electrocardiogram, and provide health care service using the measured bio-signals only when specific bio-signal measurement devices are provided, so that there is inconvenience in the use of the devices by a user without hindering his daily life and in the reception of health care service through the devices.

### SUMMARY OF THE INVENTION

It is, therefore, an object of the present invention to provide a system for measuring bio-signals which is capable of measuring and providing bio-signals using a necklace-type measurement device and a method of providing health care service using the system.

Another object of the present invention is to provide a system for measuring bio-signals which is capable of providing health care information service using bio-signals measured and provided by using a necklace-type bio-signal measurement device and a method of providing health care service using the system,.

In accordance with a preferred embodiment of the present invention, there is provided a bio-signal measurement system for measuring bio-signals and providing relevant health care service, including a bio-signal measurement device formed in a necklace shape, and configured to measure various bio-signals, perform signal processing on the measured bio-signals, and transmit the processed bio-signals through a short-range wireless communication module; and a portable terminal device configured to perform signal processing on the transmitted bio-signals, store the processed bio-signals, calculate relevant bio-indexes, provide a monitoring service related to the bio-signals, compare the calculated bio-indexes with previously stored bio-indexes, and selectively provide a relevant health care information service.

In accordance with another preferred embodiment of the present invention, there is provided a method of providing health care service using a bio-signal measurement system for measuring and providing bio-signals, the method including, when an operational key of a necklace-type bio-signal measurement device is entered, supplying power to respective elements and measuring various bio-signals; performing analog signal processing and digital signal conversion on the measured various bio-signals, and transmitting the processed bio-signals to a portable terminal device through a short-range wireless communication module; performing signal processing on the transmitted bio-signals, and calculating bio-indexes corresponding to the bio-signals; providing a monitoring service related to the bio-signals using the bio-signals and the bio-indexes; and comparing the bio-indexes with previously stored bio-indexes and selectively providing a health care information service.

Unlike the prior art methods of measuring a bio-signal using a specific bio-signal measurement device and providing health care service using the measured bio-signal, the present invention is configured to, when the operational key of the necklace-type bio-signal measurement device is entered, supply power to respective elements, measure various bio-signals, perform analog signal processing and digital signal conversion on the measured bio-signals, transmit the processed bio-signals to the portable terminal device through the short-range wireless communication module, perform signal processing on the transmitted bio-signals, calculate bio-indexes corresponding to the bio-signals, provide a monitoring service related to the bio-signals by using the bio-signals and the bio-indexes, and provide a health care information service by comparing the bio-indexes with previously stored bio-indexes. Accordingly, since bio-signals can be automatically measured using the necklace-type bio-signal measurement device without hindering a user's daily life and health care service using the bio-signals can be effectively provided, thereby increasing the convenience of use by the user.

### BRIEF DESCRIPTION OF THE DRAWINGS

The above and other objects and features of the present invention will become apparent from the following description of preferred embodiments given in conjunction with the accompanying drawings, in which:

FIG. 1 is a block diagram of a system for measuring bio-signals that is suitable for the provision of health care service using bio-signals measured through a necklace-type bio-signal measurement device according to the present invention;

FIG. 2 is a diagram showing an example of the bio-signal measurement system, including a necklace-type bio-signal measurement device, according to the present invention;

FIG. 3 is a diagram showing various necklace-type bio-signal measurement devices according to the present invention; and

FIG. 4 is a flow chart showing a process of providing a health care service using bio-signals measured through the necklace-type bio-signal measurement device.

### DETAILED DESCRIPTION OF THE PREFERRED EMBODIMENTS

The technical gist of the present invention resides in a process of, when the operational key of a necklace-type bio-signal measurement device is entered, supplying power to respective elements, measuring bio-signals by respective sensors, performing signal processing on the bio-signals, transmitting the bio-signals to a portable terminal device through short-range wireless communication, receiving and storing the bio-signals in the portable terminal device, processing the bio-signals and calculating bio-indexes, and providing health care information service for providing a warning message or a warning alarm if any of the bio-indexes is out of a relevant normal range. The problems of the prior art technologies can be overcome using the technical means of the present invention.

Hereinafter, preferred embodiments of the present invention will be described in detail with reference to the accompanying drawings so that they can be readily implemented by those skilled in the art.

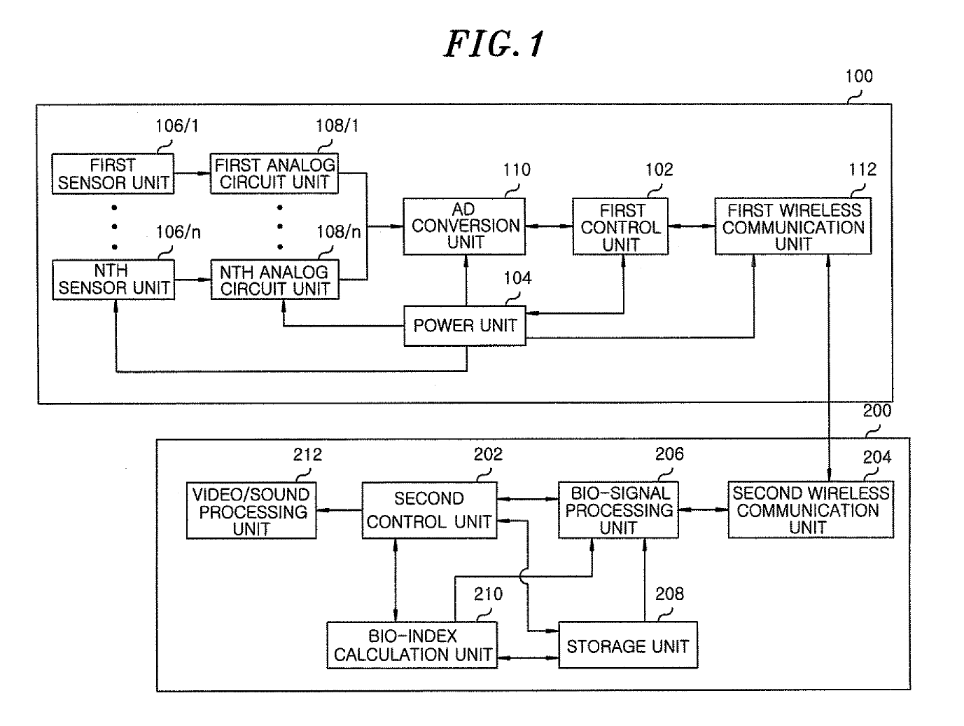

FIG. 1 is a block diagram of a system for measuring bio-signals that is suitable for the provision of health care service using bio-signals measured through a necklace-type bio-signal measurement device according to the present invention. The bio-signal measurement system includes a bio-signal measurement device 100 and a portable terminal device 200. Here, the bio-signal measurement device 100 includes a first control unit 102, a power unit 104, a first sensor unit to an nth sensor unit 106/1 to 106/n, a first analog circuit unit to an nth analog circuit unit 108/1 to 108/n, an Analog-to-Digital (AD) conversion unit 110, and a first wireless communication unit 112, and the portable terminal device 200 includes a second control unit 202, a second wireless communication unit 204, a storage unit 208, a bio-signal processing unit 206, a bio-index calculation unit 210, and a video/sound processing unit 212.

Referring to FIG. 1, when an operational key is entered, the bio-signal measurement device 100 activates respective elements thereof by supplying power to them, measures bio-signals (for example, an electrocardiogram, a respiratory signal, a body temperature, an acceleration signal, etc.) through respective sensors, performs analog signal processing on the measured bio-signals, converts the analog signals into digital signals, and sends the bio-signals, which are converted into the digital signals, to the portable terminal device 200 through a short-range wireless communication module.

The bio-signal measurement device 100 will be described in detail as follows. The first control unit 102 performs control of the overall operation of the bio-signal measurement device 100. When a user presses the operational button of the bio-signal measurement device 100 in a standby state to input an accordingly generated operational key signal, the first control unit 102 activates the bio-signal measurement device 100 by providing a control signal, instructing the power unit 104 to supply power, to the power unit 104, provides a measurement control signal to the first sensor unit to nth sensor unit 106/1 to 106/n, and provides an analog signal processing control signal, instructing the first analog circuit unit to nth analog circuit 108/1 to 108/n to perform analog signal processing on measured bio-signals, to the first analog circuit unit to nth analog circuit 108/1 to 108/n.

Furthermore, the first control unit 102 provides a control signal, instructing the AD conversion unit 110 to convert the analog bio-signals into digital signals, to the AD conversion unit 110, receives bio-signals converted into the digital signals accordingly, and performs control so that the digital bio-signals are transmitted to the portable terminal device 200 through the first wireless communication unit 112.

When a power supply control signal is provided by the first control unit 102 in response to the input of the operational key signal, the power unit 104 supplies power to the first sensor unit to nth sensor unit 106/1 to 106/n, the first analog circuit unit to nth analog circuit unit 108/1 to 108/n, the AD conversion unit 110 and the first wireless communication unit 112.

Thereafter, the first sensor unit to nth sensor unit 106/1 to 106/n include sensors that measure an electrocardiogram, a respiratory signal, a body temperature, an acceleration signal, etc. In response to a measurement control signal from the first control unit 102, the first sensor unit to nth sensor unit 106/1 to 106/n measure bio-signals through the various measurement sensors, and transfer the bio-signals to the first analog circuit unit to nth analog circuit unit 108/1 to 108/n.

Furthermore, the first analog circuit unit to nth analog circuit unit 108/1 to 108/n include amplifiers and filters, and perform analog signal processing. In response to an analog signal processing control signal from the first control unit 102, the first analog circuit unit to nth analog circuit unit 108/1 to 108/n performs analog signal processing in such a way as to amplify and filter bio-signals, such as an electrocardiogram, a respiratory signal, a body temperature, an acceleration signal, etc., sent from the first sensor unit to nth sensor unit 106/1 to 106/n, and transmits the resulting signals to the AD conversion unit 110.

The AD conversion unit 110 converts analog signals into digital signals. In response to a signal conversion control signal from the first control unit 102, the AD conversion unit 110 converts the bio-signals, transmitted from the first analog circuit unit to nth analog circuit unit 108/1 to 108/n, into digital signals, and transmits the digital bio-signals to the first control unit 102.

Thereafter, the first wireless communication unit 112 performs short-range wireless communication, for example, Body Area Network (BAN)-based wireless communication, including Bluetooth and Zigbee. Under the control of the first control unit 102, the first wireless communication unit 112 performs wireless data communication (for example, the transmission of bio-signals) between the bio-signal measurement device 100 and the portable terminal device 200.

Meanwhile, the portable terminal device 200 includes a mobile communication terminal, a PDA, etc., and provides a monitoring service for monitoring bio-signals and bio-indexes and health care information service. The portable terminal device 200 receives the transmitted bio-signals from the bio-signal measurement device 100 through the short-range wireless communication module, processes the signals, stores the signals, measures bio-indexes for the signals, checks whether any of the measured bio-indexes falls within a normal range, and provides health care information service for providing a warning message or a warning alarm if the bio-index is out of a normal range.

The portable terminal device 200 will be described in detail as follows. The second control unit 202 controls the overall operation of the portable terminal device 200. When bio-signals are received through the second wireless communication unit 204, the second control unit 202 provides a signal processing control signal to the bio-signal processing unit 206, provides a control signal, instructing the bio-index calculation unit 210 to calculate bio-indexes for the bio-signals, to the bio-index calculation unit 210, checks whether each of the calculated bio-indexes falls within a normal range previously stored in the storage unit 208, and provides a control signal, instructing the video/sound processing unit 212 to display a relevant warning message or issue a warning alarm, to the video/sound processing unit 212 if the calculated bio-index does not fall within a normal range, that is, the calculated bio-index is out of a normal range. In this case, the second control unit 202 can provide monitoring service related to the bio-signals (for example, the display of bio-signals, or the display of bio-indexes) using the provided bio-indexes.

Furthermore, the second wireless communication unit 204 performs short-range wireless communication, for example, BAN-based wireless communication, including Bluetooth and Zigbee. Under the control of the second control unit 202, the second wireless communication unit 204 performs wireless data communication (for example, the transmission of bio-signals) between the bio-signal measurement device 100 and the portable terminal device 200.

Thereafter, in response to a signal processing control signal from the second control unit 202, the bio-signal processing unit 206 performs predetermined signal processing on the bio-signals received from the second wireless communication unit 204, stores the processed bio-signals in the storage unit 208, and transmits the bio-signals to the bio-index calculation unit 210.

Meanwhile, the storage unit 208 stores various types of application programs for the operation of the portable terminal device 200 and respective normal ranges for respective bio-indexes, so that some of them are extracted for the operation of the portable terminal device 200 or the comparison of bio-indexes and are provided to the second control unit 202.

Furthermore, in response to a bio-index calculation control signal from the second control unit 202, the bio-index calculation unit 210 calculates bio-indexes, such as a pulse rate, a respiratory rate, the amount of activity, and a body temperature by using bio-signals (for example, an electrocardiogram, a respiratory signal, a body temperature, and an acceleration signal) transmitted from the bio-signal processing unit 206, and transmits the calculated bio-indexes to the second control unit 202. The bio-indexes are stored and managed in the storage unit 208.

Meanwhile, the video/sound processing unit 212 includes a display device, such as a Liquid Crystal Display (LCD), and an output device, such as a speaker, and displays a warning message related to health care information service, provided from the second control unit 202, on the display device, such as an LCD, or issues a warning alarm through the output device, such as a speaker.

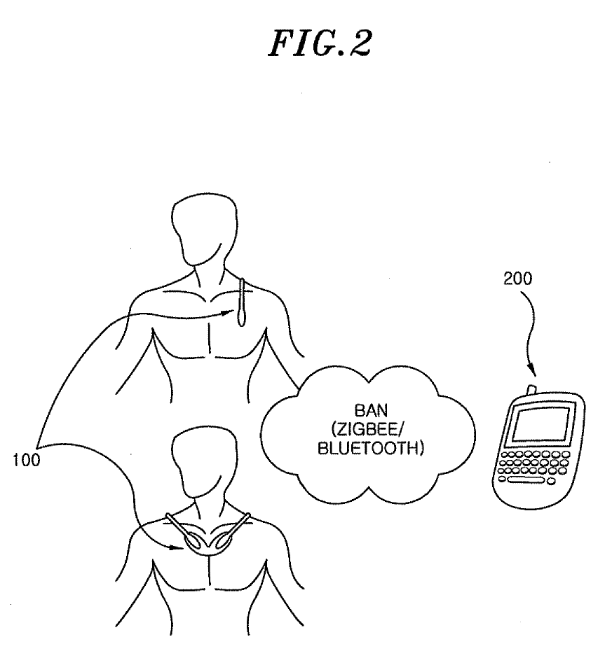

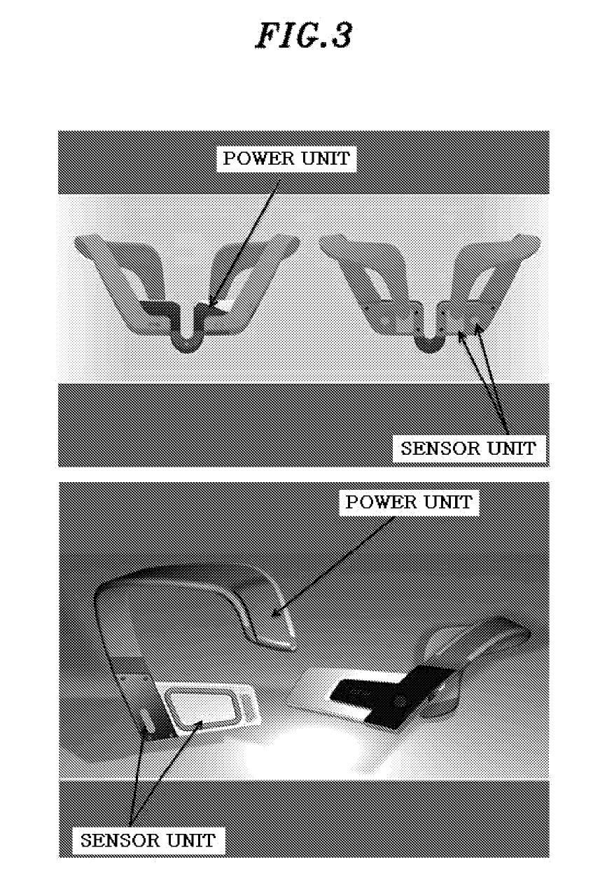

As an example of the above-described bio-signal measurement system, FIG. 2 is a diagram showing an example of the bio-signal measurement system, including a necklace-type bio-signal measurement device, according to the present invention, and FIG. 3 is a diagram showing various necklace-type bio-signal measurement devices according to the present invention. From FIGS. 2 and 3, it can be seen that it is possible to measure bio-signals and then send the bio-signals to the portable terminal device by one of various necklace-type bio-signal measurement devices and to perform monitoring service and health care information service related to the bio-signals.

In detail, it is possible to perform analog signal processing on bio-signals measured by the necklace-type bio-signal measurement device, convert the bio-signals into digital signals, send the digital bio-signals to the portable terminal device through the short-range wireless communication module, and provide monitoring service and health care information service related to bio-signals through the processing of the bio-signals and the calculation of bio-indexes.

Now, a process of, in the bio-signal measurement system having the above-described construction, supplying power in response to the entry of the operational key, measuring bio-signals by using the sensors, performing analog signal processing on the measured bio-signals, performing digital signal conversion on the bio-signals, transmitting the bio-signals to the portable terminal device, and providing a health care information service for issuing an alarm if any of the bio-signals is out of a relevant normal range through the processing of the bio-signals and the calculation of bio-indexes will be described below.

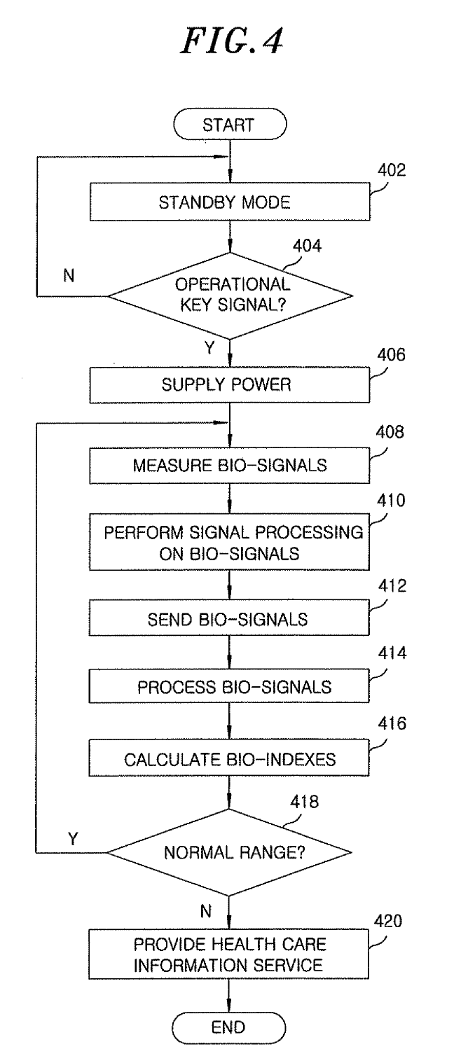

FIG. 4 is a flow chart showing a process of providing a health care service using bio-signals measured through the necklace-type bio-signal measurement device.

Referring to FIG. 4, when the bio-signal measurement system, including the necklace-type bio-signal measurement device 100 and the portable terminal device 200, is in a standby mode (step 402), the first control unit 102 checks whether an operational key signal, generated when a user presses the operational button of the bio-signal measurement device 100, is entered (step 404).

If, as a result of the checking at step 404, the operational key signal has been entered, the first control unit 102 provides a power supply control signal to the power unit 104, and thus the power unit 104 supplies power to the first sensor unit to nth sensor unit 106/1 to 106/n, the first analog circuit unit to nth analog circuit unit 108/1 to 108/n, the AD conversion unit 110 and the first wireless communication unit 112 (step 406).

Meanwhile, the first sensor unit to nth sensor unit 106/1 to 106/n measure bio-signals by using the sensors for measuring an electrocardiogram, a respiratory signal, a body temperature and an acceleration signal, and transmit the measured bio-signals to the first analog circuit unit to nth analog circuit unit 108/1 to 108/n (step 408).

Thereafter, the first analog circuit unit to nth analog circuit unit 108/1 to 108/n perform analog signal processing on bio-signals for an electrocardiogram, a respiratory signal, a body temperature, an acceleration signal, etc., transmitted from the first sensor unit to nth sensor unit 106/1 to 106/n, in such a way as to amplify them using amplifiers and filter them using various filters, and transfer the processed bio-signals to the AD conversion unit 110, and the AD conversion unit 110 converts the bio-signals into digital signals and transmits them to the first control unit 102 (step 410).

The first control unit 102 transmits the digital bio-signals, transmitted from the AD conversion unit 110, to the portable terminal device 200 through the first wireless communication unit 112 (step 412).

Meanwhile, the bio-signals, received by the second wireless communication unit 204 of the portable terminal device 200, are transmitted to the bio-signal processing unit 206, and the bio-signal processing unit 206 performs predetermined signal processing on the bio-signals, stores them in the storage unit 208 and transmits them to the bio-index calculation unit 210 (step 414).

The bio-index calculation unit 210 calculates bio-indexes, such as a pulse rate, a respiratory rate, the amount of activity, and a body temperature, using the bio-signals (for example, an electrocardiogram, a respiratory signal, a body temperature, and an acceleration signal) transmitted from the bio-signal processing unit 206, and transmits the calculated bio-indexes to the second control unit 202 (step 416). In this case, the bio-indexes, calculated by the bio-index calculation unit 210, may be stored and managed in the storage unit 208.

Meanwhile, the second control unit 202 checks whether any of the transferred bio-indexes falls within a relevant normal range by comparing the bio-index, transmitted from the bio-index calculation unit 210, with a relevant bio-index extracted from and previously stored in the storage unit 208 (step 418). In this case, the second control unit 202 may provide monitoring service (for example, the display of bio-signals or the display of bio-indexes) using the transmitted bio-indexes.

If, as a result of the checking at step 418, any of the bio-indexes falls within a relevant normal range, step 408 and its subsequent steps are continuously performed to provide the health care service of the bio-signal measurement system. In contrast, if any of the bio-indexes does not fall within a relevant normal range, the second control unit 202 provides a control signal, instructing the video/sound processing unit 212 to provide a relevant warning message or warning alarm, to the video/sound processing unit 212, so that the video/sound processing unit 212 performs a health care information service for displaying a warning message on the display device, such as an LCD, or a health care information service for issuing a warning alarm through the output device, such as a speaker, (step 420). Of course, both a warning message and a warning alarm may be provided at the same time.

Accordingly, it is possible to perform analog signal processing and digital signal processing on bio-signals measured using the necklace-type bio-signal measurement device through the sensors, transmit the processed bio-signals to the portable terminal device, provide a monitoring service related to the bio-signals through the processing of the bio-signals and the calculation of bio-indexes, and selectively provide a health care information service through the comparison of the bio-indexes with previously stored bio-indexes.

While the invention has been shown and described with respect to the preferred embodiments, it will be understood by those skilled in the art that various changes and modifications may be made without departing from the spirit and scope of the invention as defined in the following claims.

What is claimed is:

1. A bio-signal measurement system for measuring bio-signals and providing relevant health care service, comprising:
a bio-signal measurement device formed in a necklace shape, and configured to measure various bio-signals, perform signal processing on the measured bio-signals, and transmit the processed bio-signals through a short-range wireless communication module; and
a portable terminal device configured to perform signal processing on the transmitted bio-signals, store the processed bio-signals, calculate relevant bio-indexes, provide a monitoring service related to the bio-signals, compare the calculated bio-indexes with previously stored bio-indexes, and selectively provide a relevant health care information service.

2. The bio-signal measurement system of claim 1, wherein the bio-signal measurement device includes:
a plurality of sensor units for measuring and sending the various bio-signals;
a plurality of analog circuit units for performing analog signal processing on the sent bio-signals;
an Analog-to-Digital (AD) conversion unit for converting the analog bio-signals into digital signals;
a first wireless communication unit for transmitting the converted bio-signals to the portable terminal device through the short-range wireless communication module; and
a first control unit for controlling the measurement of the various bio-signals, the processing of the analog signals, the conversion into the digital signals, and the transmission of the bio-signals.

3. The bio-signal measurement system of claim 2, wherein the bio-signal measurement device further includes a power unit for supplying power to respective elements under control of the first control unit when an operational key signal is input to the first control unit.

4. The bio-signal measurement system of claim 3, wherein each of the plurality of analog circuit units has an amplifier for amplifying the bio-signals and a filter for filtering the bio-signals.

5. The bio-signal measurement system of claim 4, wherein the short-range wireless communication module is a wireless communication module based on a Body Area Network (BAN), including Bluetooth and Zigbee.

6. The bio-signal measurement system of claim 1, wherein the portable terminal device includes:
a second wireless communication unit for receiving the bio-signals, transmitted from the bio-signal measurement device, via the short-range wireless communication module;
a bio-signal processing unit for performing signal processing on the received bio-signals;
a bio-index calculation unit for calculating bio-indexes corresponding to the signal-processed bio-signals;
a second control unit for controlling the reception of the bio-signals, the signal processing of the bio-signals, the calculation of the bio-indexes, the provision of the monitoring service using the bio-signals and the bio-indexes, and the selective provision of the health care information service through comparing the bio-indexes with the previously stored bio-indexes; and
a video/sound processing unit for outputting at least one of a warning message and a warning alarm under selective control of the health care information service.

7. The bio-signal measurement system of claim 6, wherein the portable terminal device further includes a storage unit for storing and managing the bio-signals and the bio-indexes.

8. The bio-signal measurement system of claim 7, wherein the bio-signals include one or more of an electrocardiogram, a respiratory signal, a body temperature, and an acceleration signal.

9. The bio-signal measurement system of claim 8, wherein the bio-indexes include one or more of a pulse rate, a respiratory rate, an amount of activity, and a body temperature, which correspond to the bio-signals.

10. A method of providing health care service using a bio-signal measurement system for measuring and providing bio-signals, the method comprising:
supplying power to respective elements and measuring various bio-signals, when an operational key of a necklace-type bio-signal measurement device is entered;
performing analog signal processing and digital signal conversion on the measured various bio-signals, and transmitting the processed bio-signals to a portable terminal device through a short-range wireless communication module;
performing signal processing on the transmitted bio-signals, and calculating bio-indexes corresponding to the bio-signals;
providing a monitoring service related to the bio-signals by using the bio-signals and the bio-indexes; and
comparing the bio-indexes with previously stored bio-indexes and selectively providing a health care information service.

11. The method of claim 10, wherein the performing analog signal processing is performed by amplifying the bio-signals and filtering the bio-signals.

12. The method of claim 11, wherein the short-range wireless communication module is a wireless communication module based on a Body Area Network (BAN), including Bluetooth and Zigbee.

13. The method of claim 12, wherein the providing monitoring service is performed in such a way of displaying the bio-signals and the bio-indexes.

14. The method of claim 10, wherein the providing the health care information service is performed when one of the bio-indexes does not fall within a normal range for the previously stored bio-indexes.

15. The method of claim 14, wherein the providing the health care information service is performed in such a way of providing at least one of a warning message and a warning alarm if the bio-index does not fall with the normal range.

16. The method of claim 15, wherein the bio-signals include one or more of an electrocardiogram, a respiratory signal, a body temperature, and an acceleration signal.

17. The method of claim 16, wherein the bio-indexes include one or more of a pulse rate, a respiratory rate, an amount of activity, and a body temperature, which correspond to the bio-signals.
